(19) United States
(12) Patent Application Publication
(10) Pub. No.: US 2010/0120887 A1
(43) Pub. Date: May 13, 2010

(54) SUSTAINED RELEASE FORMULATION OF MELATONIN

(75) Inventors: Michael TERMAN, New York, NY (US); John Charles HAKALA, Lakewood, CO (US)

Correspondence Address: LAW OFFICES OF ALBERT WAI-KIT CHAN, PLLC, 141-07 20th AVENUE, WORLD PLAZA, SUITE 604, WHITESTONE, NY, 11357, US

(73) Assignee: THE TRUSTEES OF COLUMBIA UNIVERSITY IN THE CITY OF NEW YORK, New York, NY (US)

(21) Appl. No.: 12/623,961

(22) Filed: Nov. 23, 2009

Related U.S. Application Data
(63) Continuation-in-part of application No. PCT/US2008/064734, filed on May 23, 2008.
(60) Provisional application No. 60/940,240, filed on May 25, 2007.
(60) Provisional application No. 60/940,009, filed on May 24, 2007.

(51) Int. Cl. A61K 31/4045; A61P 25/00; A61P 5/00

(52) U.S. Cl. 514/419

(57) ABSTRACT

The present invention relates to low-dose formulations of melatonin, and methods of use thereof, which provide a sustained release of melatonin so as to rapidly increase plasma levels of melatonin, maintain a relatively high level (which mimics the endogenous level of a young subject) for approximately 5-6 hours, and then decrease so as to achieve low levels by early morning (rapid washout), thereby avoiding a “hangover effect”. The sustained release formulations of the invention may be used to treat a variety of sleep-related disorders, including, but not limited to, delayed onset and maintenance forms of insomnia.

### CROSS REFERENCES TO RELATED APPLICATIONS

This application is a Continuation-in-part of International Application No. PCT/US2008/064734, filed May 23, 2008 which claims benefit of U.S. Ser. No. 60/940,009 filed May 24, 2007 and U.S. Ser. No. 60/940,240 filed May 25, 2007, the entire content of which is incorporated by reference into this application.

### FIELD OF THE INVENTION

The present invention provides for sustained release formulations of melatonin and their use in the treatment of sleep disorders.

### BACKGROUND OF THE INVENTION

The action of pineal acetone extracts in blanching tadpole skin was first demonstrated in 1917. In 1958 Lerner and colleagues reporting isolation of the active principal from beef pineal as N-acetyl-5-methoxytryptamine, or “melatonin” (Lerner et al., 1958). Wurtman, Axelrod and colleagues showed that melatonin is synthesized in the mammalian pineal gland from tryptophan, by the formation of serotonin, followed by N-acetylation catalyzed by N-acetyltransferase and methylation by hydroxyindole-O-methyltransferase.

It was soon found that melatonin levels in mammals are high at night and low in the day, thereby providing an association between melatonin and the sleep cycle. These levels are primarily regulated by suprachiasmatic nuclei-mediated inhibition of pineal melatonin production during the day and facilitation of melatonin production during the night (Kalsbeek et al., 2000). Melatonin is inactivated by hydroxylation to 6-hydroxymelatonin by the P450 oxidase enzyme system, and about 85 percent is excreted in urine and feces as the sulfate conjugate, 6-sulfatoxymelatonin (Arendt, 1995). The half-life of melatonin is brief with bolus formulations producing physiological levels for one to two hours (Waldhauser et al., 1984; Aldhous et al., 1985). Levels are dose-dependent and conventionally far higher than physiological levels. Even with short half-life, high doses can maintain supraphysiologic doses longer than two hours.

Although U.S. prevalence estimates of chronic insomnia vary widely—often based solely on subjective measures—a NIH Workshop on Neurobiology of Sleep and Waking offered a conservative estimate that 10 percent of the adult population suffers from a form of insomnia. The National Sleep Foundation's “Sleep in America Poll” found a far higher frequency of adults reporting insomnia of at least a few nights a week (58 percent) with 10 percent using prescribed medications and 14 percent using over the counter sleep aids.

Symptom amelioration by prescription hypnotics carries the hazards of dependency and even mortality (Kripke, 1998). Use of melatonin offers appeal as an alternative, and its FDA classification as a dietary supplement has made consumer access simple, inexpensive and widespread, although access outside the U.S. remains restricted. Both immediate and sustained release formulations (“IR” and “SR”, respectively) are commercially available, 3 mg being the more typical dose, although quality control has been lacking, and dose variability and presence of contaminants is prevalent (Naylor, 1999), and basic pharmacokinetic properties have not been consistently established.

The standard melatonin formulation, sold in 3 mg doses, is characterized by fast systemic release and metabolism, due to the short half-life of the melatonin molecule. Thus, these formulations provide for a spike in melaton into supraphysiologic levels 1-2 hours after ingestion, and then a gradual return to normal levels. Such immediate release formulations therefore fail to mimic the time course and amplitude of endogenous melatonin, the secretion of which is synchronized with the duration of the biological night. Limited data on commercially available SR formulations show that exogenous melatonin can remain in the system for up to 8 hours after wakeup time, thereby raising the issue of carry-over daytime soporific (or “hangover”) effects. Further, when bioavailability is extended into the morning hours, melatonin can create a phase-delay in the circadian clock which ultimately can lead to or exacerbate insomnia at bedtime.

Over the past decade there have been several small studies of exogenous melatonin administration to treat insomnia in adults. Dosing has ranged over orders of magnitude (0.2 to 100 mg; see MacFarlane et al., 1991; Hughes et al., 1998; Monti et al., 1999; and Zhdanova et al., 2001). Although the lack of adverse effects (Seabra et al., 2000) is reassuring, reports of pharmacokinetic characteristics have been inconsistent. There are a number of patents and patent applications relating to melatonin and its uses, including U.S. Pat. No. 6,703,412; U.S. Pat. No. 6,214,377, U.S. Pat. No. 5,498,423, U.S. Pat. No. 5,449,683, U.S. Pat. No. 5,430,029, U.S. Pat. No. 5,242,941, and United States Patent Application Publication No. US 2004/0248966A1.

### SUMMARY OF THE INVENTION

The present invention relates to low-dose formulations of melatonin, and methods of use thereof, which provide a sustained release (“SR”) of melatonin so as to rapidly increase plasma levels of melatonin, maintain a relatively high level (which mimics the endogenous level of a young adult subject) for approximately 5-6 hours, and then decrease so as to achieve low levels by early morning (rapid washout), thereby avoiding a “hangover effect”. The SR formulations of the invention may be used to treat a variety of sleep-related disorders, including, but not limited to, delayed onset and maintenance forms of insomnia. The timing of the rise of melatonin levels to aid in falling asleep, the maintenance of elevated levels to promote uninterrupted sleep, and the morning washout to avoid morning somnolence, thereby promoting a “well rested” feeling, are all advantages of the methods and formulations of the invention.

### BRIEF DESCRIPTION OF THE FIGURES

FIG. 1 shows the typical salivary melatonin onset curves for 9 individuals (3 pg/mL threshold).

FIG. 2 shows the effect of darkness (top) vs. light (bottom) in the middle of the night

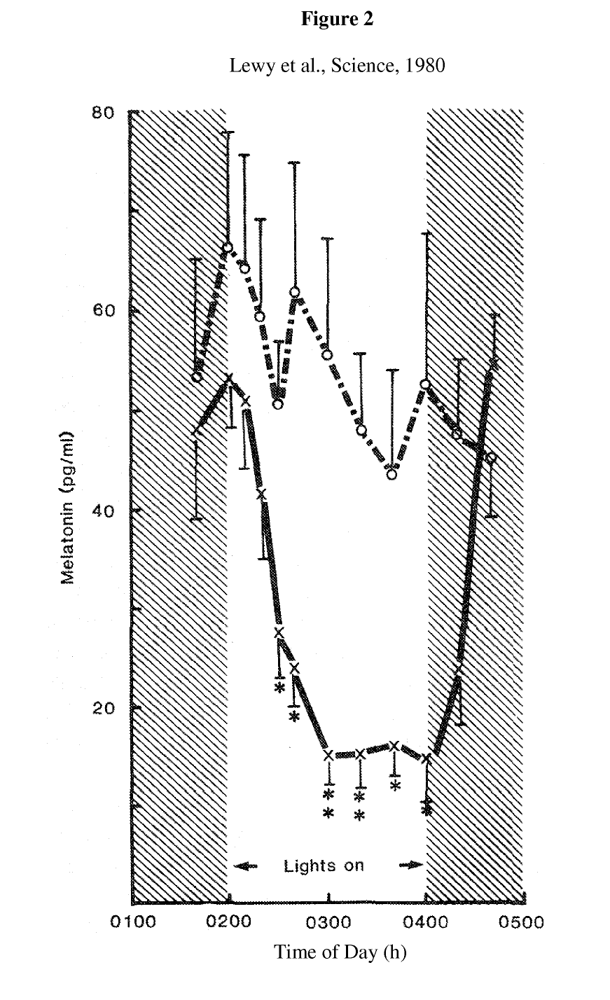

FIGS. 3 shows the following: FIG. 3A: Changes in total plasma melatonin as a function of time of day. FIG. 3B: Levels of ingested, as compared with endogenous, melatonin, as a function of time of day.

FIG. 4 shows the following: FIG. 4A: “Rapid washout” of melatonin in an individual. FIG. 4B: “Slow washout” of melatonin in an individual.

FIG. 5 shows the following: FIG. 5A: Average levels of 6-sulfatoxymelatonin in urine of individuals over time after treatment with 0.2 mg melatonin, 2.0 mg melatonin, or placebo tablets at 9 PM. FIG. 5B: Adjusted levels of 6-sulfatoxymelatonin subtracting the endogenous component (placebo levels) from the endogenous component (associated with the 0.2 or 2.0 mg dose).

### DETAILED DESCRIPTION OF THE INVENTION

The following terms shall be used to describe the present invention. In the absence of a specific definition set forth herein, the terms used to describe the present invention shall be given their common meaning as understood by those of ordinary skill in the art.

As used herein, the expression “sustained release formulation” refers to a composition that gradually introduces a substance into blood circulation without the spiking of immediate release formulations, and maintains bioavailability for several hours without rapid decline, preceding final washout.

### Formulations

Melatonin may be purchased from commercial sources, may be synthesized (see, for example, U.S. Reissue Pat. No. RE35,631), or may be purified from a natural source. In particular non-limiting embodiments of the invention, the melatonin is micronized, for example such that 80 percent or 90 percent of the particles have a diameter of less than 20 microns or preferably less than 10 microns.

The amount of melatonin used in the formulations of the invention may be between about 0.05 and 2 mg (“about” meaning a variation of up to 20 percent of the recited value), or between about 0.05 and 1.5 mg, or between about 0.05 and 1 mg, preferably between about 0.05 and 0.5 mg, or between about 0.05 and 0.25 mg, or between about 0.05 and 0.15 mg, or about 0.1 mg, or about 0.15 mg, or about 0.2 mg. The SR formulations of the invention are compressed tablets, preferably comprising one or more binder compound. In particular, non-limiting embodiments, the amount of binder compound(s) may be between about 20 and 80 percent, or between about 30 and 70 percent of the total weight of the tablet. In particular, non-limiting embodiments, the amount of binder compound(s) may be between about 40 and 60 percent of the total weight of the tablet. It may be noted that administration of a dose of 2.0 mg in healthy middle aged subjects was not “washed out” by the next morning, but such a dose may be required in certain individuals due to variations in body mass index, first-pass excretion effect, liver metabolic rate, treatment with other medications, time of day of tablet ingestion, or age (children have higher endogenous levels relative to adults and may require a higher dosage).

In non-limiting embodiments of the invention, the binder may be a cellulose ether such as methylcellulose or hydroxypropyl methylcellulose (e.g. Methocel K100M USP (Dow Chemical Co., Midland, Mich.), hydroxypropyl cellulose, microcrystalline cellulose (e.g., silicified microcrystalline cellulose, such as ProSolv (by Penwest Pharmaceuticals, Patterson, N.Y.), which is silicified microcrystalline cellulose and colloidal silicon dioxide), polyvinyl pyrrolidone (povidone), povidone (polyvinyl pyrrolidone), povidone crosprovidone lactose blend, mannitol, sorbitol, sucrose, other compressible sugar, or other binder known in the art, where binders formulated for sustained-release uses are preferred. In preferred non-limiting embodiments of the invention, a SR tablet according to the invention comprises a cellulose ether such as methylcellulose or hydroxypropylmethyl cellulose or microcrystalline cellulose, or a mixture of cellulose ether and microcrystalline cellulose.

The SR formulations of the invention may optionally comprise further ingredients, for example one or more oil, one or more wax, and/or silicon dioxide.

In particular, non-limiting embodiments of the invention, a SR tablet according to the invention comprises: a melatonin dose as set forth above (accounting for less than 10 percent, preferably less than 5 percent, of the weight of the tablet); a binder composition comprising between 40-60 percent weight of the tablet, and preferably about 50 percent weight of the tablet, of a mixture of methylcellulose and microcrystalline cellulose (where preferably about 10-20% of the total weight of the tablet is methylcellulose). In related embodiments, the SR tablet further comprises between 30-50 percent weight silicon dioxide, between about 2-12 percent weight oil, and/or between about 2-12 percent weight wax. Further optionally, the tablet may be coated with a material known in the art, for example a coating that facilitates swallowing. In non-limiting embodiments, the total weight of the SR formulation of the invention may be between about 50 and 500 mg, preferably between 100 and 350 mg.

In a specific, non-limiting embodiment, the present invention provides for a SR tablet comprising a dose of melatonin as set forth above, 10.0 mg safflower oil, USP (Spectrum Chemical, Gardenia, Calif.), 10.0 mg carnauba wax (Strahl & Pitsch, W. Babylon, N.Y.), 100 mg Micosolle (Biomicotec, Torrance, Calif.), 37.8 mg Methocel K100M, USP (Dow Chemical Co., Midland, Mich.), and 88.2 mg ProSolv (Penwest Pharmaceuticals, Patterson, N.Y.), compressed into a tablet weighing about 250 mg.

### Methods of Treatment

Conditions which may be treated according to the invention include but are not limited to, sleeplessness (including delayed sleep onset insomnia and sleep maintenance insomnia, and Primary Insomnia as defined by the DSM-IV: a “predominant complaint of difficulty initiating or maintaining sleep, or nonrestrorative sleep, for at least one month”), interrupted (light) sleep, early morning awakening, difficulty awakening, circadian rhythm disorders, sleep disorder associated with depression, hypertension, shift work/daytime sleep-related disorders; jet lag, sleep disorder associated with Alzheimer's disease, sleep disorder associated with Parkinson's disease, sleep disorder associated with schizophrenia, sleep disorder in geriatric patients, developmental brain disorder-related sleep disturbances, anxiety-related sleep disorders, sleep disorders associated with metabolic disorders, sleep disorders caused by pharmacologic agents, sleep disorder associated with Adult Attention Deficit Hyperactivity Disorder and Autistic Disorder, and reduced or absent endogenous melatonin production resulting from a disorder or ablation of the pineal gland. In addition, the methods of the invention may be used in conjunction with morning light therapy to treat any of the foregoing disorders.

Treatment means a reduction in the symptoms and signs of any of the above-listed disorders, including subjective improvement and improved quality of life. For example, and not by way of limitation, reduction in symptoms and signs of sleep-related disorders may be identified using the Pittsburgh Sleep Quality Index (Buysse et al., 1989) or the St. Mary's Hospital Sleep Questionnaire (Leigh et al., 1988). In specific non-limiting embodiments of the invention, treatment may constitute an increase in the average number of hours of sleep per night, for example, by at least one hour, at least two hours, at least three hours, at least four hours, or at least five hours. In addition or alternatively, treatment may constitute decreasing sleep onset latency by at least 30 minutes, at least one hour, or at least two hours, and/or may result in a sleep onset latency of less than 30 minutes, less than one hour, or less than two hours.

Accordingly, the present invention provides for methods of treating a disorder as set forth above, e.g. a sleep-related disorder, comprising administering, to a subject in need of such treatment, a melatonin SR tablet formulated as set forth in the preceding section. The method may be practiced on an as-needed basis or as part of a regimen which may be practiced one or more times per day. A regimen may, in non-limiting embodiments of the invention, continue for up to 5 days, up to one week, up to one month, up to 2 months, up to six months, up to one year, or for at least 5 days, at least one week, at least two weeks, at least one month, at least 6 months. It is understood that in a regimen lasting for a week or more, one or two doses per week may be missed.

In a specific non-limiting embodiment of the invention, for patients who wish to treat interrupted (light) sleep without a shift in sleep timing, a SR dose of melatonin according to the invention may be given about 2-3 hours before a desired (or habitual) bedtime. The habitual bedtime of a subject is that time of day at which, on average (±45 minutes) the subject goes to bed, for example, but not by way of limitation, based on all days of the week or, alternatively, work days.

In another specific non-limiting embodiment of the invention, for patients with delayed sleep onset and delayed awakening, a SR dose of melatonin according to the invention may be administered approximately 5-6 hours before habitual bedtime, in order to phase-advance the circadian clock signals for sleep onset and awakening. Preferably, but not by way of limitation, bright light exposure should be avoided following pre-sleep melatonin administration, for example for a range of between about 2-6 hours. This may be achieved, for example but not by way of limitation, by wearing filtered sunglasses (such as wrap around “blue blockers”).

In yet another specific non-limiting embodiment of the invention, to phase delay sleep onset, a subject, having slept earlier in the day, may be administered melatonin at the about the habitual bedtime.

In another specific non-limiting embodiment of the invention, the SR dose may be administered between about 10-14 hours, or between about 10-12 hours, before a desired wakeup time.

The SR formulation of the invention is administered orally.

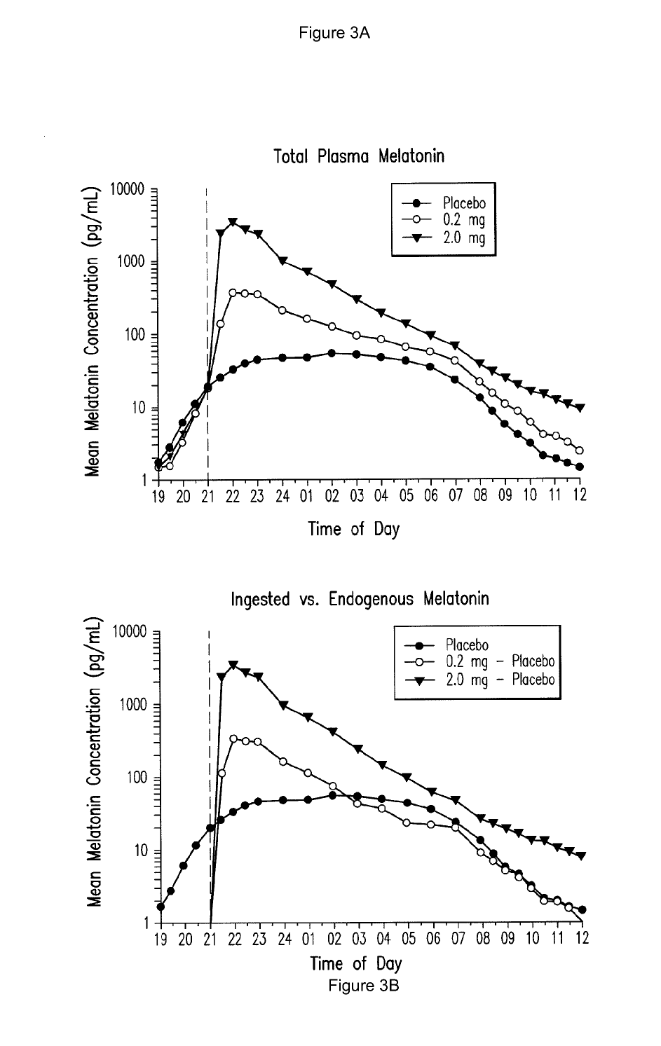

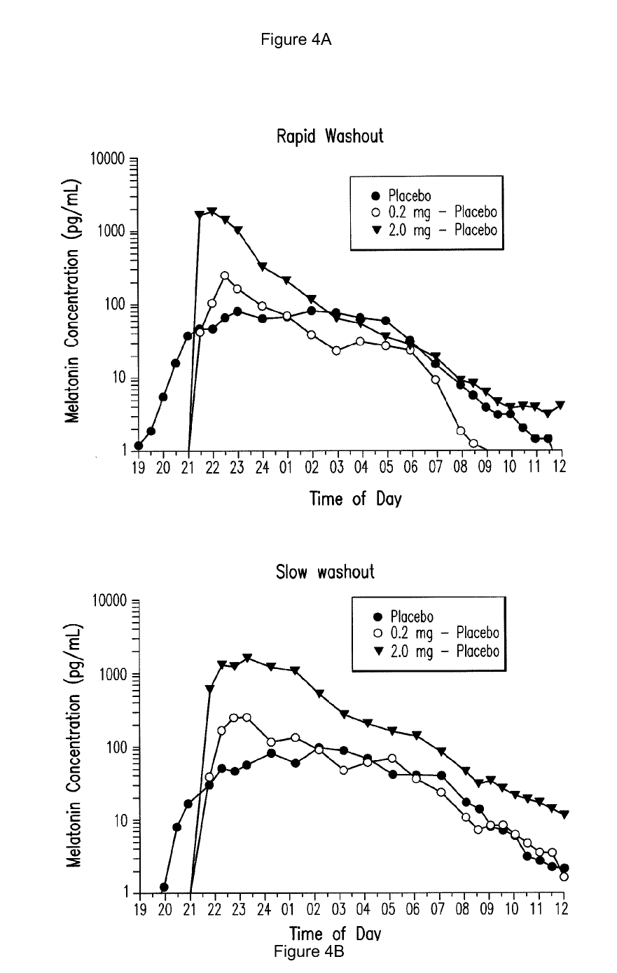

Average curves, such as those shown in FIG. 3AB, are not always consistent with the elimination rates of melatonin in particular individuals. For example, FIG. 4A-B shows examples of elimination rates for two subjects with relatively rapid (FIG. 4A) and slow (FIG. 4B) washout, respectively.

“Slow washout” is defined herein as residual circulating exogenous melatonin 12-15 mg after tablet ingestion. Determination of residual levels requires reference to endogenous levels of melatonin, measured without tablet administration, at the corresponding 12-15 hour time point. A patient with relatively slow washout, for example, would be better treated with 0.1 mg melatonin than, for example, 0.2 mg. For this reason, it may be desirable to provide a dosage formulation which permits the administration of a lower dose, for example, a scored tablet with one or two score marks. In a specific, non-limiting embodiment, a 0.2 mg tablet may be scored to allowing easy downward dosage to 0.1 mg (by breaking the tablet in half). Clinically, such dosage adjustment could be based on the observation of morning hangover or direct detection of high residual levels of the urinary metabolite, 6-hydroxymelatonin sulfate (aMT6S), around noontime. A patient with slow washout at 0.2 mg would show residual aMT6S at noontime, which would serve as a guide to lower dosing. A “residual amount” of aMT6s would be considered, for example, an aMT6S level of above about 10 micrograms. Accordingly, the present invention provides for a method of adjusting the dosage of melatonin comprising measuring the level of urinary aMT6S at about noontime (preferably between 10:30 AM and 1:30 PM, or between 11 AM and 1 PM), wherein, if the level is above about 10 micrograms after correcting for the endogenous level separately measured at that time point, the dosage of melatonin is decreased by at least about 25 percent or at least about 50 percent.

The present invention provides a method of treating a sleep disorder in a subject, comprising administering to a subject in need of such treatment a sustained release formulation of melatonin comprising between about 0.05 mg to about 2 mg melatonin. In one embodiment, the sustained release formulation is in a form of tablet. In one embodiment, the formulation of melatonin is administered between about 2 to 3 hours prior to the subject's habitual bedtime. In another embodiment, the formulation of melatonin is administered between about 5 to 6 hours prior to the subject's habitual bedtime. In yet another embodiment, the formulation of melatonin is administered at about the subject's habitual bedtime. In yet another embodiment, the formulation of melatonin is administered between about 10 to 14 hours prior to the subject's wakeup time.

In another embodiment, the above method can be used in treating Advanced Sleep Phase Disorder (ASPD), in which sleep onset is uncontrollably earlier than the normal range, e.g., 6 PM. In such case, the melatonin formulation could be used to delay—rather than to advance—the circadian clock by ingestion (upon brief awakening) in the middle of the subject's sleep episode (e.g., 10 PM), thus introducing the substance during the latter half of the night and extending through the morning. The effect would be enhanced by remaining in dim light throughout the morning or wearing blue-blockers.

In another embodiment, the present method can be used for treating bipolar disorder (manic depression), in which the internal clock shifts earlier during manic phases, one result being premature awakening after brief sleep. Administering the melatonin formulation of the present invention in the latter half of the night (with or without the use of blue-blockers) might serve to attenuate or shorten the manic phase. Melatonin has been termed ‘the physiological signal of darkness’. Placing a patient in darkness for up to 14 hours (an extended night) can break the manic episode with reduced need for antipsychotic drugs (Bipolar Disord. 2005 February; 7(1):98-101. Dark therapy for mania: a pilot study. Barbini B, Benedetti F, Colombo C, Dotoli D, Bernasconi A, Cigala-Fulgosi M, Florita M, Smeraldi E.). Using the melatonin formulation of the present invention would have the same effect without the need for extended dark exposure.

In one embodiment, the formulation of melatonin in the above method is administered orally. Alternatively, the formulation of melatonin may be administered by injection or nasal spray, or administered by patch. In one embodiment, the formulation of melatonin is administered at a dose of about 0.2 mg melatonin. Furthermore, the formulation of melatonin may comprise between about 30-50 percent weight silicon dioxide. In another embodiment, the formulation of melatonin further comprises between about 2-12 percent weight oil. In yet another embodiment, the formulation of melatonin further comprises between about 2-12 percent wax. In yet another embodiment, the melatonin is in micronized form prior to incorporation into the tablet.

In one embodiment, the dosage of melatonin used in the above method can be adjusted by a method comprising the step of measuring the level of urinary 6-hydroxymelatonin sulfate at about noontime, wherein the dosage of melatonin is decreased by at least about 25 percent when the level of the detected 6-hydroxymelatonin sulfate is higher than that detected in a subject not treated by the formulation of melatonin. In another embodiment, the dosage of melatonin is decreased by at least about 50 percent.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Throughout this application, various references or publications are cited. Disclosures of these references or publications in their entireties are hereby incorporated by reference into this application in order to more fully describe the state of the art to which this invention pertains.

### Example 1

### Emhanced Melatonin Formulation

Circadian timing varies widely between people. This is most obvious by measuring the level of pineal melatonin in circulation throughout the evening, when production begins. Within the normal range, “larks” may show onset as early as 18:00, “owls” as late as 01:00. In delayed sleep phase disorder (DSPD), onset may be as late as 05:00. Sleep adjusts commensurately, causing insomnia in owls and DSPD, incompatible with the workday.

Errors with Current Melatonin Administration

To normalize late sleep timing, melatonin taken before sleep in mega doses—the prevalent method—is no solution. In immediate-release form (1-10 mg), it creates a supraphysiologic spike after pineal melatonin onset, which quickly washes out (t_{1/2}≈45 min). There is no professional consensus that this is effective as a sleep aid, despite OTC availability in the US. In marketed controlled-release forms, the blood level almost surely remains high well into the next day (pharmacokinetics mostly not reported), with three potential side effects: (a) hangover; (b) delaying (instead of advancing) action on the circadian clock; and (c) photosensitization of the retina.

To the extent that pre-sleep melatonin ingestion aids sleep onset and continuity, literature indicates that lower doses in the physiologic range (0.1-0.3 mg) are more effective than mega doses.

To move the circadian clock earlier to support natural sleep onset, a non-soporific physiological dose is need as much as 3 hours before endogenous onset and 5 hours before delayed sleep onset. Thus far, this has been demonstrated only with repeated 0.1-mg immediate-release tablets taken throughout the late afternoon/evening; clinically infeasible.

### Melatonin Deficiency

In addition to its action on the circadian clock, some people—especially elderly—have trouble sleeping because of reduced melatonin. In this case, literature suggests that replenishing melatonin throughout the night is therapeutic.

Immediate-release forms are contraindicated because of fast washout. The

European prescription controlled-release form (2 mg Circadin®, Lundbeck, for insomnia age 50+)) produces supraphysiologic levels with morning overshoot, and is not well tuned for this purpose.

### Risk of Melatonin Suppression by Nighttime Light

Light exposure during the night immediately suppresses melatonin production, and causes activation/sleeplessness and undesired shifts in the circadian clock. It may also pose a long-term hazard for breast and prostate cancer. Shift workers are the primary risk group.

Light restriction on the night shift is impractical. Melatonin supplementation on the night shift is an active preventive prospect, but current dosing protocols (mega doses, slow controlled release) are not the solution.

The present invention provides a controlled-release, pharmaceutical-grade formulation of melatonin that: (a) matches but does not exceed youthful blood levels, (b) gradually washes out by early morning, (c) does not exert a soporific effect when taken before pineal melatonin onset, (d) elicits circadian phase advances when taken before pineal melatonin onset, (e) can be used before bedtime to replenish deficient melatonin levels, and (f) can be timed according to an individual's circadian phase (the owl-lark dimension) to optimize effect.

### Example 2

### Dissolution Rates

3 mg SR tablets were prepared as follows. Melatonin was a gift from Neurim Pharmaceuticals, S.A., Switzerland. For preparation of the SR formulations of the invention, the melatonin was micronized such that 90 percent of the micronized drug was less than 10 microns in diameter. 3.0 mg micronized melatonin was combined with 10.0 mg safflower oil, USP (Spectrum Chemical, Gardenia, Calif.), 10.0 mg carnauba wax (Strahl & Pitsch, W. Babylon, N.Y.), 100 mg Micosolle (Biomicotec, Torrance, Calif.), 37.8 mg Methocel K100M, USP (Dow Chemical Co., Midland, Mich.), and 88.2 mg ProSolv (Penwest Pharmaceuticals, Patterson, N.Y.). This mixture was compressed into an oval tablet measuring 0.225″×0.535″ and weighing 249 mg.

Dissolution of the SR tablets was studied using a Type 3 Dissolution Apparatus-VanKel BioDis II custom designed with 12 rows. The dissolution of 4 tablets per vessel was studied. Media for 1 hour study was 0.1 N Hal (pH 1.2); for the 2 hour study was 0.05 M acetate buffer (pH 4.5), and for the 3-10 hour study was 0.05 M phosphate buffer (pH 6.8). The temperature of the solutions was 37 degrees C. (±0.5 degree). Speed was 7 dips/min. Sample times were 1, 2, 3, 4, 5, 6, 7, 8, 9, and 10 hours. Absorbance was measured at 278 nm. Results are tabulated in Table 1 at the end of this section.

It was noted that, during dissolution, the 4 tablets in each dissolution cell remained separate. Thus, there was no apparent clumping or adhering that could confound the release pattern of each tablet.

Column 7 of Table 1 shows that the release rate of melatonin from the 3 mg SR tablets was observed to be about 30 percent in the first hour, about 10 percent for each of the next three hours, and then at a decreased rate over the next six hours. Column 10 of Table 1 shows that half of the melatonin was released between 2-3 hours, with the remainder released over the following 7-8 hours. The variation in release rate appeared to be quite low. It should be noted, however, that as each sample consisted of multiple tablets, this is not a precise determination of variation.

Since a large percentage of melatonin was released within the first hour, a more detailed examination of release within that first hour, at 5, 15, 30, and 45 minutes, with 1 3.0 mg SR tablet placed into 200 ml 0.1 N HCl, and the conditions otherwise the same as above. The results are presented in Table 2, at the end of this section. It was observed that melatonin was released slowly within the first hour, with only 6 percent released in the first 15 minutes, an additional 4 percent released in the next 15 minutes, an additional 3 percent released in the third 15 minute interval, and (based on Table 1) an additional 17 percent released in the final 15 minutes of the first hour. Thus, the release of melatonin from the SR tablet was associated with a small lag time for release.

TABLE 1

| Time | Media | Sample | Absorbance | mg dissolved | % dissolved | Avg % dissolved | % RSD | Cum % | Cum Avg % | SD |
|---|---|---|---|---|---|---|---|---|---|---|
| 1 hr | 0.1 N HCl | 1 | 0.4812 | 3.75 | 31 | | | 31 | | |
| | | 2 | 0.4174 | 3.40 | 28 | 30 | 5.78 | 28 | 30 | 2 |
| | | 3 | 0.4531 | 3.77 | 31 | | | 31 | | |
| 2 hr | pH 4.5 | 1 | 0.2441 | 1.99 | 17 | | | 48 | | |
| | | 2 | 0.2083 | 1.70 | 14 | 15 | 8.15 | 42 | 46 | 3 |
| | | 3 | 0.2199 | 1.79 | 15 | | | 46 | | |
| 3 hr | pH 6.8 | 1 | 0.1953 | 1.59 | 13 | | | 61 | | |
| | | 2 | 0.1680 | 1.37 | 11 | 11 | 15.41 | 54 | 57 | 4 |
| | | 3 | 0.1433 | 1.17 | 10 | | | 56 | | |
| 4 hr | pH 6.8 | 1 | 0.1627 | 1.32 | 11 | | | 72 | | |
| | | 2 | 0.1385 | 1.13 | 9 | 10 | 12.12 | 63 | 67 | 5 |
| | | 3 | 0.1290 | 1.05 | 9 | | | 65 | | |
| 5 hr | pH 6.8 | 1 | 0.1423 | 1.16 | 10 | | | 82 | | |
| | | 2 | 0.1237 | 1.01 | 8 | 9 | 11.47 | 72 | 75 | 6 |
| | | 3 | 0.1137 | 0.83 | 8 | | | 73 | | |
| 6 hr | pH 6.8 | 1 | 0.1158 | 0.94 | 8 | | | 90 | | |
| | | 2 | 0.1026 | 0.83 | 7 | 7 | 11.08 | 79 | 82 | 6 |
| | | 3 | 0.0829 | 0.76 | 6 | | | 79 | | |
| 7 hr | pH 6.8 | 1 | 0.0870 | 0.71 | 6 | | | 96 | | |
| | | 2 | 0.0822 | 0.67 | 6 | 6 | 5.04 | 84 | 56 | 7 |
| | | 3* | 0.0787 | 0.64 | 5 | | | 84 | | |
| 8 hr | pH 6.8 | 1* | 0.0716 | 0.58 | 5 | | | 100 | | |
| | | 2* | 0.0577 | 0.47 | 4 | 4 | 12.93 | 88 | 92 | 7 |
| | | 3* | 0.0576 | 0.47 | 4 | | | 88 | | |
| 9 hr | pH 6.8 | 1* | 0.0534 | 0.43 | 4 | | | 104 | | |
| | | 2* | 0.0452 | 0.37 | 3 | 3 | 9.12 | 91 | 95 | 7 |
| | | 3* | 0.0465 | 0.38 | 3 | | | 91 | | |
| 10 hr | pH 6.8 | 1* | 0.0416 | 0.34 | 3 | | | 107 | | |
| | | 2* | 0.0363 | 0.30 | 2 | 3 | 10.61 | 94 | 98 | 8 |
| | | 3* | 0.0341 | 0.28 | 2 | | | 94 | | |

*out of linearity range

TABLE 2

| | | | | cum | Average | | |
|---|---|---|---|---|---|---|---|
| | | | mg | % | cum % | Std. | % |
| Time | Sample | Absorbance | dissolved | dis'd | dis'd | Dev. | RSD |
| 5 | 1* | 0.0170 | 0.14 | 2 | | | |
| | 2* | 0.0188 | 0.15 | 3 | 2 | 0 | 6.10 |
| | 3* | 0.0169 | 0.14 | 2 | | | |
| 15 | 1* | 0.0435 | 0.34 | 6 | | | |
| | 2* | 0.0532 | 0.42 | 7 | 6 | 1 | 9.93 |
| | 3* | 0.0485 | 0.38 | 6 | | | |
| 30 | 1* | 0.0721 | 0.55 | 9 | | | |
| | 2 | 0.0880 | 0.67 | 11 | 10 | 1 | 9.91 |
| | 3 | 0.0823 | 0.63 | 10 | | | |
| 45 | 1 | 0.0962 | 0.72 | 12 | | | |
| | 2 | 0.1145 | 0.86 | 14 | 13 | 1 | 9.02 |
| | 3 | 0.1107 | 0.83 | 14 | | | |

*out of linearity range

### Example 3

### In Vivo Testing

Six women and four men, age 49.5±3.2 (mean±SD), with normal health and sleep patterns, were used in the following study.

0.2 mg SR and 2.0 mg SR melatonin were prepared as follows. Melatonin was a gift from Neurim Pharmaceuticals, S.A., Switzerland. For preparation of the SR formulations of the invention, the melatonin was micronized such that 90 percent of the micronized drug was less than 10 microns in diameter. Either 2.0 mg or 0.2 mg micronized melatonin was combined with 10.0 mg safflower oil, USP (Spectrum Chemical, Gardenia, Calif.), 10.0 mg carnauba wax (Strahl & Pitsch, W. Babylon, N.Y.), 100 mg Micosolle (Biomicotec, Torrance, Calif.), 37.8 mg Methocel K100M, USP (Dow Chemical Co., Midland, Mich.), and 88.2 mg ProSolv (Penwest Pharmaceuticals, Patterson, N.Y.). This mixture was compressed into an oval tablet measuring 0.225″×0.535″ and weighing approximately 250 mg (±5 mg).

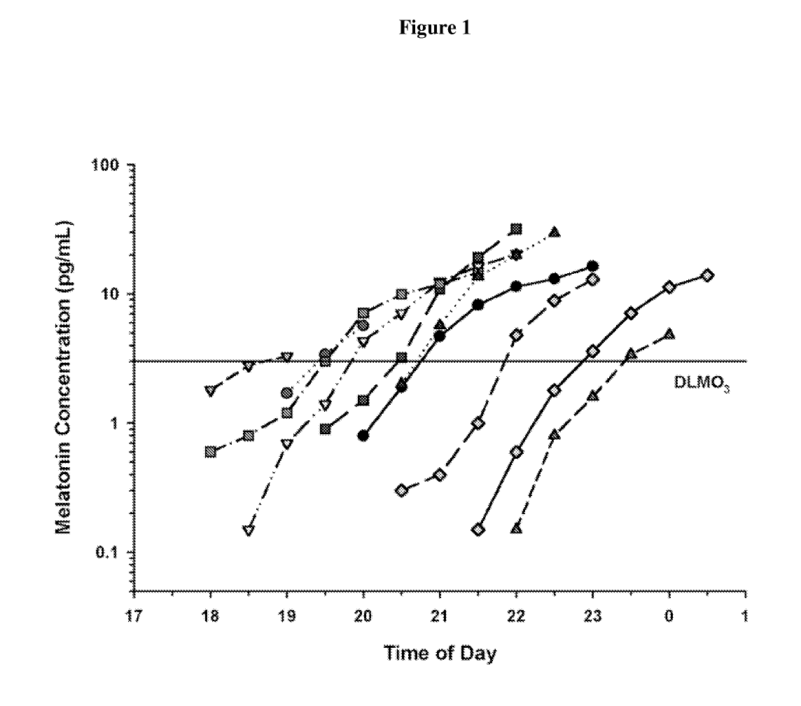

Subjects received single tablets of placebo, 0.2 mg SR melatonin, and 2.0 mg SR melatonin in a blinded, randomized manner. Hospital sessions were conducted overnight at 1-2 week intervals, and serial blood plasma samples were drawn between 7 PM-12 noon the next day. Samples were assayed for melatonin concentration. FIG. 1A-B summarizes average data for the ten subjects. FIG. 1A shows results for the three sessions with tablet administration at 9 PM. FIG. 1B shows results for endogenous pineal melatonin secretion (as assessed on the placebo night); for comparison with corrected dose curves that exclude the endogenous from the exogenous component.

In vitro dissolution studies indicated that dissolution followed an exponential approaching to a maximum with 82 percent dissolved at 6 hours and 98 percent dissolved at 10 hours. For in vivo testing under placebo, the average endogenous melatonin curve rose from 1.7 pg/ml at 1900 hours to 19.3 pg/ml at 2100 h (the time of tablet administration). Peak levels reaching 54 pg/ml were maintained between 2200-0600 h, followed by an exponential washout completed by 1200 h (t_{1/2}≈71 minutes). Melatonin absorption and elimination followed a complex pattern of rapid rise to peak concentration from 2130-2300 h (0.2 mg, 328 pg/ml; 2.0 mg, 3467 pg/ml) followed by slow, parallel exponential declines. Under 0.2 mg, the curve superposed on the endogenous washout curve starting at 0700 h (t_{1/2}≈73 minutes). The curve associated with the 2.0 mg dose remained at greater than 10 pg/ml until 1200 h.

The results show that, for these middle-aged subjects, the endogenous melatonin peak amplitude (mean=58 pg/ml) was lower than that of younger subjects (-100-300 pg/ml). Both doses of SR melatonin produced smoothly rising and falling melatonin concentrations. The peak levels (for the 0.2 mg SR dose, 274 pg/ml, and for the 2.0 mg SR dose, 3251 pg/ml) were separated by approximately one log unit, mirroring the doses. Similarly, the areas under the curves were separated by approximately one log unit (for 0.2 mg SR, 1603 pg/ml0.30 min; for 2.0 mg SR, 13,831 pg/ml0.30 min).

The 0.2 mg SR dose was found to achieve a satisfactory approximation to the peak melatonin amplitude typically seen in young adults, up to 300 pg/ml. The ingested melatonin curves differed in shape from the endogenous curve, mainly in the gradual decline beginning 2 hours after ingestion. Endogenous melatonin tends to remain at an asymptotically high level several hours longer, before washout begins. However, the curve shape of nocturnal melatonin production varies widely between individuals, and is not considered to be functionally significant.

The subjects showed melatonin levels (corrected for endogenous levels) above 10 pg/ml for approximately 11 hours with the 0.2 mg SR dose, and 13 hours with the 2.0 mg SR dose. The washout tails of the endogenous and 0.2 mg SR curves converge in the early morning hours. In contrast, the curve associated with the 2.0 mg SR dose remained relatively high at the end of the test run, failing to wash out by noon.

In conclusion, the 0.2 mg SR formulation was found to provide essentially a physiological dose of melatonin with concentrations ≧10 pg/ml lasting 8.7±2.1 hours after ingestion. Taken 2 hours before bedtime, washout was observed to coincide with that of endogenous melatonin. This formulation may be used as a supplement taken around the time of melatonin onset for people with low endogenous melatonin production and for those whose sleep would benefit by late afternoon or early evening administration as a circadian phase-advancing agent.

### Example 4

### Varied Washout in Patients

FIG. 4A-B shows examples of elimination rates for two subjects with relatively rapid (FIG. 4A) and slow (FIG. 4B) washout, respectively.

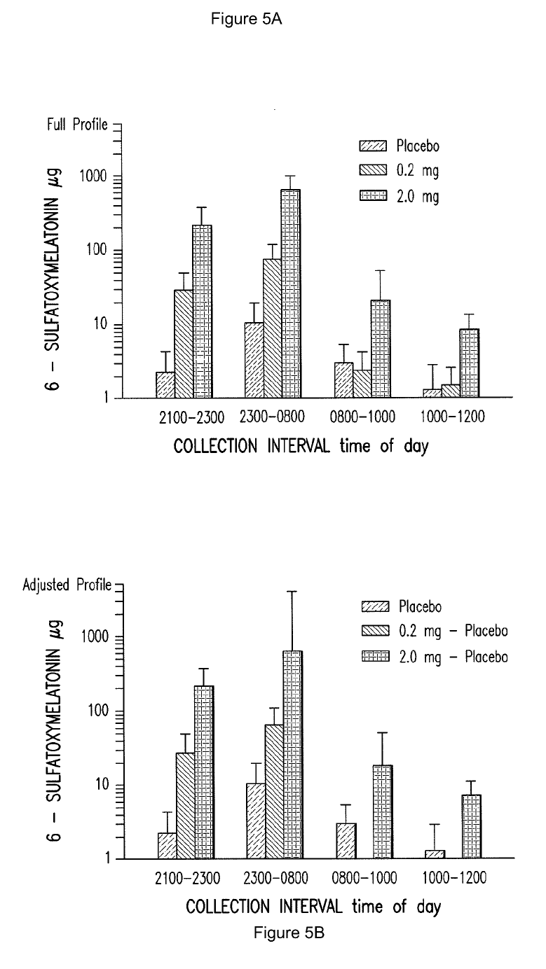

FIG. 5A-B illustrates the results when urinary 6-sulfatoxymelatonin (aMT6S) was measured in subjects over the two hours after tablet ingestion, overnight, and from 8-10 AM and 10 AM-12 PM. On average, there was no residual aMT6S in the final 0.2 mg sample at 12 PM, in contrast to the 2.0 mg sample.

### Example 5

### Initial Clinical Evaluation

The present invention describes tablet formulation, in vitro and in vivo pharmacokinetics in a healthy, middle-aged subject group, and a range of potential uses. In the past three years, the present invention has been used in open clinical treatment of a variety of cases comprising circadian sleep disorders and depression. The strategy for treatment concentrates on serious cases, from which application to less severe cases—far more common population-wide—can be inferred. For example, in Delayed Sleep Phase Disorder (DSPD), the patient cannot fall asleep before wee hours of the night or early morning because of misalignment of the internal circadian timing system with external day and night. Standard sleep medications do not correct the problem, because they are not chronobiotics. Melatonin is the chronobiotic par exemplar. Milder circadian misalignment is highly prevalent in the population, often starting in adolescence. A typical example is the worker who must maintain a 9-5 schedule but cannot fall asleep before 1 AM (“early insomnia”), leaving six hours for sleep availability, leading to daytime fatigue, disorientation, lapses of alertness, and weekend oversleeping that can trigger depressed mood. Such people comprise a primary market for the present invention, with the prospect of reduced reliance on sleeping pills.

Thus far, an ensemble of chronotherapeutic interventions has been used in clinical trials. Melatonin acts synergistically with light therapy at the opposite time of day to advance the circadian clock, which makes the combination strategy useful especially for patients with clinical depression accompanied by delayed sleep phase.

Tests of the present invention are ongoing, but with very promising results for people with “simpler” insomnia:

Case 1: A female attorney age 47 was required to work into the evening as late as 10 PM in a brightly lit office pool. She could not fall asleep until 2 AM after arriving home at 11 PM. The target was to prepare her for sleep by midnight by advancing her circadian clock. She took the tablet at 7 PM, after which she wore blue-blocking amber glasses to reduce the delaying effect of fluorescent office light exposure in the evening. Within days she was able to “calm down” quickly after work and sleep a full night starting at 12 AM.

Case 2: A female postgraduate fellow age 30 experienced seasonal depression each winter, when she developed overwhelming pressure to sleep long hours, but could not fall asleep before 1 AM. Since she spent evenings at home under normal room light, she did not require blue-blockers. Melatonin at 6:30 PM, coupled with light therapy for 45 minutes upon awakening, allowed her to go to sleep at 11:30 PM with remission of depressive symptoms.

Case 3: A female patient age 19, with a history of seasonal depression that had caused her to drop out of college for periods, was seen in September before symptoms set in. As is typical for college students, she would keep very late hours on some nights, and compensate on others. In order to forestall expected relapse in October, she began using the melatonin every evening around 9 PM, with intermittent light therapy for 15-30 minutes upon wake-up. She continued to allow herself occasional late nights (2 AM sleep onset, elective, not due to insomnia), was able to sleep at midnight without effort, and maintained high mood and productivity into the winter.

Case 4: Patient J. M. male, age 28, an accomplished scholar, had experienced extreme delayed sleep phase disorder since adolescence, aggravated in recent years with sleep onset around 7 AM and waking around 3 PM. The distinct downside for him was the inability to collaborate with colleagues during the normal workday. He resisted hypnotic medication but intermittently used alcohol to fall asleep a few hours earlier. The phase-advancing strategy combined 3 chronotherapeutic methods: (a) 0.2 mg controlled release melatonin62 in the evening; (b) blue-blocking (400-535 nanometers) glasses until sleep onset; and (c) 1 hour of light therapy at 10,000 lux upon awakening. Melatonin was initially taken too early (about 7 hours before sleep onset) because the patient insisted he was ready to fall asleep earlier; after stabilization, the delay to sleep was about 4-5 hours. He followed a 1-3 day schedule of 30-minute advances in wake-up time and light therapy, based on his level of confidence that he would not oversleep. Sleep onset remained spontaneous throughout. He achieved the goal of sleep from 11:30 PM to 7 AM in about two weeks, and expressed incredulity that this could happen.

The present invention has been described as an optimum approach “under development” in Chronotherapeutics for Affective Disorders, the first clinical treatment manual to endorse this method.

### REFERENCES

Aldhous et al., 1985, Br. J. Clin. Pharmacol. 19:517-521.
Arendt, 1995, “Melatonin and the mammalian pineal gland.” Chapman & Hall, London.
Basel, S., 2009, “Chronotherapeutics for Affective Disorders: A Clinician's Manual for Light and Wake Therapy”, Karger, 1:70-71
Buysse et al., 1989, Psychiatry Res. 28:193-213.
Dawson et al., 1998, J. Biol. Rhythms 13:532-538.
Garfinkel et al., 1995, Lancet 346:541-544.
Haimov et al., 1995, Sleep 18:598-603.
Haimov and Lavie, 1995, Drugs Aging 7:75-78.
Hughes et al., 1998, Sleep 21:52-68.
James et al., 1990, Neuropsychopharmacol. 3:19-23.
Kalsbeek et al., 2000, Eur. J. Neurosci. 12:3146-3154.
Kripke, 1998, Biol. Psychiatry 43:687-693.
Leigh et al., 1988, Sleep 11(5):448-453.
Lerner et al., 1958, J. Am. Chem. Soc. 80:2587.
MacFarlane et al., 1991, Biol. Psychiatry 30:371-376.
Monti et al., 1999. Arch. Gerontol. Geriatr. 28:85-98.
Naylor et al., 1999, Adv. Exp. Med. Biol. 467:769-777.
Seabra et al., 2000, J. Pineal Res. 29:193-200.
Waldhauser et al., 1984, Neuroendocrinol. 39:307-313.
Wurtman and Zhdanova, 1995, Lancet 346:1491.
Zhdanova et al., 1999, Soc. Neurosci. Abstr. 25, pt. 1:26.
Zhdanova, 2001, J. Clin. Endocrinol. Metab. 10:4727-4730.

What is claimed is:

1. A method of treating a sleep disorder in a subject, comprising administering to a subject in need of such treatment a sustained release formulation of melatonin comprising between about 0.05 mg to about 2 mg melatonin.

2. The method of claim 1, wherein the sustained release formulation is in a form of tablet.

3. The method of claim 1, wherein the formulation of melatonin is administered between about 2 to 3 hours prior to the subject's habitual bedtime.

4. The method of claim 1, wherein the formulation of melatonin is administered between about 5 to 6 hours prior to the subject's habitual bedtime.

5. The method of claim 1, wherein the formulation of melatonin is administered at about the subject's habitual bedtime.

6. The method of claim 1, wherein the formulation of melatonin is administered between about 10 to 14 hours prior to the subject's wakeup time.

7. The method of claim 1, wherein the formulation of melatonin is administered at about the middle of the subject's sleep episode.

8. The method of claim 7, wherein the method further comprises the step of keeping the subject in dim light throughout the morning or wearing blue-blockers.

9. The method of claim 1, wherein the formulation of melatonin is administered orally.

10. The method of claim 1, wherein the formulation of melatonin is administered at a dose of about 0.2 mg melatonin.

11. The method of claim 1, wherein the formulation of melatonin further comprises between about 30-50 percent weight silicon dioxide.

12. The method of claim 1, wherein the formulation of melatonin further comprises between about 2-12 percent weight oil.

13. The method of claim 1, wherein the formulation of melatonin further comprises between about 2-12 percent wax.

14. The method of claim 2, wherein the melatonin is in micronized form prior to incorporation into the tablet.

15. The method of claim 1, wherein the dosage of melatonin can be adjusted by a method comprising the step of measuring the level of urinary 6-hydroxymelatonin sulfate at about noontime, wherein the dosage of melatonin is decreased by at least about 25 percent when the level of the detected 6-hydroxymelatonin sulfate is higher than that detected in a subject not treated by the formulation of melatonin.
